(19) United States
(12) Patent Application Publication
(10) Pub. No.: US 2024/0382555 A1
(43) Pub. Date: Nov. 21, 2024

(54) PEPTIDES WITH ANTI-ANGIOGENIC ACTIVITY

(71) Applicant: CHEIRONTECH S.r.l., Napoli (IT)

(72) Inventors: Francesca CACCURI, Brescia (IT); Arnaldo CARUSO, Brescia (IT)

(73) Assignee: CHEIRONTECH S.r.l., Napoli (IT)

(21) Appl. No.: 18/690,146

(22) PCT Filed: Sep. 8, 2022
(86) PCT No.: PCT/EP2022/074974

§ 371 Date: Mar. 7, 2024

(30) Foreign Application Priority Data
Sep. 9, 2021 (IT) 102021000023357

(51) Int. Cl. A61K 38/06; A61K 38/07; A61P 9/00

(52) U.S. Cl. CPC A61K 38/06; A61K 38/07; A61P 9/00

(57) ABSTRACT

The present invention relates to a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid, for use as a drug, particularly for use in the treatment of a disorder resulting from pathological angiogenesis, and a pharmaceutical composition comprising it together with at least one pharmaceutically acceptable excipient.

### FIELD OF INVENTION

The present invention relates to peptides with anti-angiogenic activity, and a pharmaceutical composition comprising them that are useful in the treatment of angiogenesis-related disorders, such as, for example, cancer, chronic inflammation, and neo-vascularization disorders.

### STATE OF THE ART

Angiogenesis is the complex process of blood vessel formation. The process involves both biochemical and cellular events, including (i) activation of endothelial cells (ECs) by angiogenic stimulus; (ii) degradation of extracellular matrix, invasion of activated ECs into surrounding tissues, and migration to the source of the angiogenic stimulus; and (iii) proliferation and differentiation of ECs to form new blood vessels.

The control of angiogenesis is a highly regulated process involving angiogenic stimulators and inhibitors. In humans and healthy animals, angiogenesis occurs in specific and limited situations. For example, angiogenesis is normally observed in fetal and embryonic development, in the development and growth of normal tissues and organs, in wound healing, and in the formation of the corpus luteum, endometrium, and placenta.

In certain diseases, the control of angiogenesis is impaired, and thus so-called pathologic angiogenesis occurs, that is, the formation of excess or unwanted blood vessels that support the pathologic state and in many cases contribute to the cellular and/or tissue damage associated with these diseases.

Pathological angiogenesis plays an important role in tumor formation, as tumors need blood vessels to supply nutrients and oxygen and remove cellular wastes. At the same time, the formation of blood vessels in tumors allows cancer cells to enter the bloodstream and circulate throughout the body, generating metastases.

Tumors in which angiogenesis is important include solid tumors as well as benign tumors such as acoustic neuroma, neurofibroma, trachoma, and pyogenic granulomas. Pathologic angiogenesis is also associated with certain blood cancers such as leukemias and various acute or chronic neoplastic diseases of the bone marrow.

Pathologic angiogenesis also plays an important role in various chronic inflammatory diseases such as inflammatory bowel disease, psoriasis, sarcoidosis, and rheumatoid arthritis. The chronic inflammation that occurs in such diseases depends on the continuous formation of capillary sprouts in diseased tissue to maintain an influx of inflammatory cells. The influx and presence of inflammatory cells produce granulomas and thus maintain the chronic inflammatory state.

Angiogenesis, both normal and pathological, requires the action of one or more angiogenic factors. Such factors include, for example, angiogenin (ANG), vascular endothelial growth factor (VEGF-vascular endothelial growth factor), basic fibroblast growth factor (bFGF), acidic fibroblast growth factor (aFGF), epidermal growth factor (EGF), tumor necrosis factor-alpha (TNF-α), tumor growth factor-alpha (TGF-α), and tumor growth factor-beta (TGF-β).

The centrality of angiogenesis in the myriad of angiogenesis-related diseases has motivated the search for anti-angiogenic agents (i.e., agents that suppress or inhibit pathological angiogenesis).

Many anti-angiogenic agents have been isolated or developed. They include cartilage-derived factors, angiostatic steroids, angiostatic analogs of vitamin D, angiostatin, endostatin, and verostatin.

There are many different categories of anti-angiogenic agents that include, by way of example, agents that inhibit the action of growth factors; anti-invasive agents; and vascular disrupting agents.

### Agents That Inhibit the Action of Growth Factors Include:

(i) receptor antagonists, for example, an anti-VEGF receptor antibody as described in CA 2213833);
(ii) Protein kinase C inhibitors;
(iii) Tyrosine kinase inhibitors, for example, VEGF receptor tyrosine kinase inhibitors, as described in WO 96/40116);
(iv) Modulators of Tie-1 and/or Tie 2 receptor signaling; and
(v) inhibitors of protein expression, for example, inhibitors of VEGF expression, as described in U.S. Pat. No. 4,987,071).

### Anti-Invasion Agents Include:

(i) Matrix metalloproteinase inhibitors, for example, prinomastat (U.S. Pat. No. 5,753,653); ilomastat (WO 92/9556); marimastat (WO 94/2447); and batimastat (WO 90/5719),
(ii) Urokinase plasminogen activator receptor antagonists, for example, the compounds described in WO 96/40747 and WO 2000/001802, and
(iii) urokinase plasminogen activator inhibitors, for example, the compounds described in WO 2000/005245.

Vascular disrupting agents include combretastatin (U.S. Pat. No. 4,996,237) and the compounds described in WO 99/02166 and WO 00/40529.

Known anti-angiogenic agents include anti-angiogenesis peptides, such as those described in EP1640382A1, EP1668129A1, EP1786451A2, EP1799716A1, EP1812030A2, EP1951750A2, EP3209683A1, EP3621597A1.

WO 2004/031220 discloses a tumor-targeting peptide having the sequence DRYYNLRSK (SEQ ID NO. 6) directly or indirectly coupled to at least one effector unit for the treatment of cancer or cancer related diseases, such as a solid tumor selected from the group consisting of carcinoma, sarcoma, melanoma or metastases.

WO 2008/085828 discloses a peptide having the sequence GDRYCL bearing the “CXC” motif, which is G-X (3)-CL useful for modulating blood vessel formation in a cell, tissue or organ.

WO 00/63236 discloses a peptide having the sequence DRYLKFRPV able to modulate adhesion of a target cell to a substrate, in particular to inhibit melanoma cell attachment, by forming a physical barrier of peptide-associated substrate around a melanoma, thereby preventing its metastasis.

### SUMMARY OF THE INVENTION

The Applicant faced the problem of finding new peptides for the treatment of diseases associated with pathological angiogenesis.

Human herpesvirus 6 (HHV-6) is a β-herpesvirus that is highly prevalent in the human population. HHV-6 comprises two recognized species (HHV-6A and HHV-6B). HHV-6A/B show high genome homology and harbor the U94 gene. U94 has key functions in the virus life cycle and associated diseases, having proven or putative roles in virus replication, integration, and reactivation. During natural infection, U94 elicits an immune response, and the prevalence and extent of the anti-U94 response are associated with specific diseases. In particular, U94 may entirely mimic some effects of the virus at the cellular level, including inhibition of cell migration, induction of cytokines and HLA-G expression, and inhibition of angiogenesis, supporting a direct role of U94 in the development of HHV-6-associated diseases (Caselli E., et al., “The U94 Gene of Human Herpesvirus 6: A Narrative Review of Its Role and Potential Functions,” Cells. 2020 Dec; 9(12): 2608).

Based on these observations, the Applicant hypothesized that there must be a portion of the viral protein expressed by the U94 gene with anti-angiogenic activity, and initiated intensive research and development to identify this portion.

The viral protein expressed by the U94 gene is a sequence of 490 amino acids as shown in FIG. 6 (SEQ ID No. 1).

After extensive experimentation, the Applicant surprisingly found that the anti-angiogenic effect came from the four amino acid sequence at position 14-17 of the viral protein, namely the KDKY sequence (SEQ ID No. 2).

Continuing the experimentation, the Applicant further found that the anti-angiogenic effect was surprisingly derived from the sequence of only three amino acids at position 15-17 of the viral protein, namely the DKY sequence, and that this effect was maintained with the DRY sequence.

Therefore, in a first aspect, the present invention relates to a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid, for use as a drug.

Advantageously, the present invention relates to the peptide according to the first aspect of the present invention for use in the treatment of a disorder resulting from pathological angiogenesis, such as, for example, tumors and/or chronic inflammation and/or neo-vascular disorders.

In a second aspect, the present invention relates to a pharmaceutical composition comprising (a) a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid, and (b) at least one pharmaceutically acceptable excipient.

In a third aspect, the present invention relates to a method for the treatment of a disorder resulting from pathological angiogenesis, such as, for example, tumors and/or chronic inflammation and/or neo-vascularization disorders, in a subject in need thereof comprising the administration of an effective amount of a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid.

In a fourth aspect, the present invention relates to a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid.

### BRIEF DESCRIPTION OF THE DRAWINGS

FIG. 1 shows the results of example 1 of the experimental part of the present description.

FIG. 2 shows the results of example 2 of the experimental part of the present description.

FIG. 3 shows the results of example 3 of the experimental part of the present description.

FIG. 4 shows the results of example 4 of the experimental part of the present description.

FIG. 5 shows the results of example 5 of the experimental part of the present description.

FIG. 6 shows the sequence (SEQ ID No. 1) of 490 amino acids of the protein expressed from the U94 gene of human herpesvirus 6 (HHV-6).

### DETAILED DESCRIPTION OF THE INVENTION

In a first aspect, the present invention relates to a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid, for use as a drug.

The peptide according to the present invention can include from a minimum of 3 amino acids up to 5 amino acids, and thus consists of a peptide of 3, 4, or 5 amino acids. Advantageously, the peptide according to the present invention consists of a peptide of 3, 4 or 5 amino acids.

The peptide according to the present invention may include any of the natural amino acids listed in Table A below.

TABLE A

| | | | | | |
|---|---|---|---|---|---|
| Alanine | Ala | A | Arginine | Arg | R |
| Asparagine | Asn | N | Aspartic acid | Asp | D |
| Cysteine | Cys | C | Glutamic acid | Glu | E |
| Glutamine | Gln | Q | Glycine | Gly | G |
| Histidine | His | H | Isoleucine | Ile | I |
| Leucine | Leu | L | Lysine | Lys | K |
| Methionine | Met | M | Phenylalanine | Phe | F |
| Proline | Pro | P | Serine | Ser | S |
| Threonine | Thr | T | Tryptophan | Trp | W |
| Tyrosine | Tyr | Y | Valine | Val | V |

The peptide according to the present invention may also comprise any of the modified or unconventional amino acids known in the art, such as, for example, 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, sarcosine, N-methylisoleucine, 6-N-methyllysine, N-methylvaline, norvaline, norleucine, and ornithine.

The peptide according to various aspects of the present invention can be in the form of a modified peptide in which the N- and/or C-terminus is chemically modified or protected with organic compounds.

The term “derivative” or “derivative of” as employed in this description and the following claims in connection with a peptide according to the various aspects of the present invention means a peptide in which the N- and/or C-terminal is chemically modified or protected with an organic compound, such as, for example, phosphoryl (—PO₃²⁻), glycosyl, acyl, alkyl, carboxyl, amine, biotin, ubiquitin.

Examples of modification include phosphorylation, glycosylation, acylation (including acetylation, lauroylation, myristoylation, palmitoylation), alkylation, carboxylation, hydroxylation, glycation, biotinylation, ubiquitination and amidation.

Preferably, the peptide according to various aspects of the present invention can be modified at its N-terminus, most preferably by acylation, which includes for example acetylation, lauroylation, myristoylation, palmitoylation.

Depending on its length, the peptide according to various aspects of the present invention can be synthesized by a method well known in the art, for example, by an automated peptide synthesizer, or produced by genetic engineering technology. For example, a fusion gene encoding a fusion protein comprising a fusion partner and peptide is prepared by genetic engineering and then transformed in a host cell to express the fusion protein. Then, the peptide is cleaved and isolated from the fusion protein using a protease or compound in order to produce the desired peptide. For this purpose, a DNA sequence encoding amino acid residues that can be cleaved by a protease such as factor Xa or enterokinase, or a compound such as CNBr or hydroxylamine can be inserted between the polynucleotides encoding the fusion partner and the peptide.

Peptides according to the various aspects of the present invention can exist as stereoisomers or mixtures of stereoisomers; for example, the amino acids composing them can have L configuration, D configuration or be racemic independently of each other. Therefore, it is possible to obtain isomeric mixtures as well as racemes or diastereomeric mixtures or pure diastereomers or enantiomers, depending on the number of asymmetric carbons and which isomers or isomeric mixtures are present. The preferred structures of peptides are pure isomers, i.e., enantiomers or diastereomers. Preferred structures of peptides include amino acids that have the L configuration. Unless otherwise stated, it is understood that when it is indicated that an amino acid can be Ala, it is selected from L-Ala-, D-Ala- or racemic or nonracemic mixtures of both.

In a second aspect, the present invention relates to a pharmaceutical composition comprising (a) a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid, and (b) at least one pharmaceutically acceptable excipient.

The pharmaceutical composition of the present invention may comprise an amount of the peptide, or a derivative thereof, ranging from 0.0001% to 20% by weight, preferably from 0.0001% to 15% by weight, more preferably from 0.001% to 10% by weight, and even more preferably from 0.01% to 5% by weight relative to the total weight of the composition.

Preferably, the pharmaceutical composition of the present invention is prepared in suitable dosage forms that comprise an effective amount of at least one of the peptides described above together with at least one pharmaceutically acceptable excipient.

Examples of suitable dosage forms are tablets, capsules, coated tablets, granules, solutions, and syrups for oral administration; solutions, ointments and unguents for topical administration; medicated patches for transdermal administration; suppositories for rectal administration; and sterile injectable solutions. Other suitable dosage forms include sustained-release and liposome-based dosage forms for oral, injectable, or transdermal administration.

As described herein, the pharmaceutical composition of the present invention includes at least one of the peptides described above together with a pharmaceutically acceptable excipient, which, as used herein, includes any and all solvents, diluents, or other vehicles, dispersion or suspension aids, surface active agents, isotonic agents, thickening or emulsifying agents, preservatives, solid binders, lubricants, and the like, as suitable for the particular dosage form desired.

Some examples of materials that may serve as a pharmaceutically acceptable excipient include, but are not limited to, sugars such as lactose, glucose, and sucrose; starches such as cornstarch and potato starch; cellulose and its derivatives such as sodium carboxymethyl cellulose, ethyl cellulose, and cellulose acetate; adragosta powder; malt; gelatin; talc; excipients such as cocoa butter and suppository waxes; oils such as peanut oil, cottonseed oil; safflower oil; sesame oil; olive oil; corn oil; and soybean oil; glycols, such as propylene glycol; esters such as ethyl oleate and ethyl laurate; agar; buffering agents such as magnesium hydroxide and aluminum hydroxide; alginic acid; pyrogen-free water; isotonic salt solution; Ringer's solution; ethyl alcohol, and phosphate buffer solutions; other compatible nontoxic lubricants such as sodium lauryl sulfate and magnesium stearate; coloring agents; releasing agents; coating agents; sweetening agents; flavoring and fragrance agents; preservatives; and antioxidants.

The terms “pharmaceutically acceptable” and “physiologically acceptable” are intended to define, without any particular limitation, any material suitable for preparing a pharmaceutical composition to be administered to a living being.

Dosage forms may also contain other traditional ingredients such as: preservatives, stabilizers, surfactants, buffers, osmotic pressure regulating salts, emulsifiers, sweeteners, dyes, flavors and the like.

The pharmaceutical compositions of the present invention can be administered orally, parenterally, by inhalation spray, topically, rectally, nasally, buccally, vaginally, or through an implanted reservoir. The term parenteral as used in the present invention and the following claims includes subcutaneous, intracutaneous, intravenous, intramuscular, intraarticular, intrasynovial, intrasternal, intrathecal, intralesional, and intracranial injection or infusion techniques.

The pharmaceutical compositions of the present invention can also be administered by inhalation (spray, powder, or aerosol) or administered by implantation (e.g., surgically), for example, by means of an implantable device such as a stent.

The pharmaceutical composition dosage forms of the present invention can be prepared by techniques that are familiar to a pharmaceutical chemist and include mixing, granulation, compression, dissolution, sterilization and the like.

Advantageously, the present invention relates to the use of a peptide of length equal to or less than 5 amino acids, or a derivative thereof, comprising the sequence DKY, preferably XDKY (SEQ ID No. 7) or DKYX (SEQ ID No. 8), or the sequence DRY, preferably XDRY (SEQ ID No. 9) or DRYX (SEQ ID No. 10), where X is any amino acid, in the treatment of a disorder resulting from pathological angiogenesis.

Preferably, the aforementioned peptide and pharmaceutical compositions that include it are used in the treatment of diseases resulting from pathological angiogenesis, such as, for example, cancer and/or chronic inflammation and/or neo-vascular disorders.

Examples of tumors that can be usefully treated with the peptide and pharmaceutical composition of the present invention are solid tumors and blood cancers.

Solid tumors that can be treated with the peptide and the pharmaceutical composition of the invention include, sarcomas and carcinomas, such as, fibrous astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and benign solid tumors such as acoustic neuroma, neurofibroma, trachoma, and pyogenic granulomas.

Blood cancers such as leukemias that are amenable to treatment with the peptide and the pharmaceutical composition of the invention include, for example, acute lymphocytic leukemia and acute myelocytic leukemia (myeloblastic, promyelocytic, myelomonocytic, monocytic, and erythroleukemia); chronic leukemia (chronic myelocytic [granulocytic] and chronic lymphocytic leukemia); and polycythemia vera, lymphoma (Hodgkin's disease and non-Hodgkin's disease), multiple myeloma, Waldenström macroglobulinemia.

Specifically, the peptide and pharmaceutical composition of the present invention may be useful in the treatment of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acoustic neuroma, neurofibroma, trachoma, and pyogenic granulomas.

The pharmaceutical composition of the present invention used in the treatment of cancer may optionally comprise one or more antineoplastic agents, such as, (i) alkaloids, such as docetaxel, etoposide, trontecan, paclitaxel, teniposide, topotecan, vinblastine, vincristine, and vindesine; (ii) alkylating agents such as busulfan, improsulfan, piposulfan, aziridine, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trophosfamide, carmustine, chlorozotocin, photemustine, lomustine, nimustine, ranimustine, dacarbazine, mannomustine, mitobronitol, mitolactol, pipobroman, temozolomide; (iii) antibiotics and analogs such as aclacinomycin, actinomycin, anthramycin, azaserin, bleomycin, cactinomycin, carubicin, carzinophilin, chromomycin, dactinomycin, daunorubicin, doxorubicin, epirubicin, idarubicin, menogaril, mitomycin, mycophenolic acid, nogalamycin, olivomycins, peplomycin, pyarubicin, plicamycin, porphyromycin, puromycin, streptonigrin, streptozocin, tubercidin, zinostatin, zorubicin; (iv) antimetabolites such as denopterin, edatrexate, methotrexate, pyritrexim, pteropterin, trimetrexate, cladribine, fludarabine, 6mercaptopurine, thiamiprine, thioguanine, ancitabine, azacitidine, 6-azauridine, cytarabine, doxifluridine, emitefur, enocitabune, floxuridine, fluorouracil, gemcitabine, tegafur; L-asparaginase; (v) immunomodulators such as interferon-a, interferon-β, interferon-γ, interleukin-2, lentinan, propagermanium, PSK, roquinimex, sizofican, ubenimex; (vi) platinum complexes such as carboplatin, cisplatin, miboplatin, oxaliplatin; (vii) antineoplastic hormone or analogs such as calusterone, dromostanolone, epithiostanol, mepitiostane, testolacone, aminoglutethimide, mitotane, trilostane, bicalutamide, flutamide, nilutamide, droloxifene, tamoxifen, toremifene aminoglutethimide, anastrozole, fadrozole, formestane, letrozole, phosphestrol, hexestrol, polyestradiol phosphate, buserelin, goserelin, leuprolide, triptorelin, chlormadinone acetate, medroxyprogesterone, megestrol acetate, melengestrol; porfimer sodium; batimastar; and folinic acid.

Examples of chronic inflammatory diseases that can be usefully treated with the peptide and pharmaceutical composition of the present invention are inflammatory bowel diseases, such as Crohn's disease and ulcerative colitis, psoriasis, sarcoidosis, and rheumatoid arthritis.

Examples of neo-vascular disorders that can be usefully treated with the peptide and pharmaceutical composition of the present invention are (i) corneal disorders, such as for example, ocular acne rosacea, atopic keratitis, bacterial ulcers, chemical burns, excessive use of contact lenses, corneal graft rejection, epidemic keratoconjunctivitis, fungal ulcers, Herpes simplex infections herpes zoster infections, Kaposi's sarcoma, lipid degeneration, marginal keratolysis, mycobacterial infections, Mooren's ulcer, neovascular glaucoma and retrolental fibroplasia, pemfigoid radial keratotomy, filectenulosis, polyarteritis, protozoan infections, retinopathy of prematurity, rheumatoid arthritis, Steven Johnson's disease, superior limbic keratitis, syphilis, systemic lupus, Terrien's marginal degeneration, vitamin A deficiency and Wegener's sarcoidosis, and (ii) retinal disorders, such as for example, arterial occlusion, Behcet's disease, Best disease, chronic retinal detachment, uveitis/chronic neuritis, obstructive carotid disease, diabetic retinopathy, Eales disease, hyperviscosity syndromes, infections causing retinitis or choroiditis, Lyme disease, macular degeneration, mycobacterial infections, optic pits, Paget's disease, post-laser complications, presumed ocular histoplasmosis, pseudoxanthoma elasticum, retinopathy of prematurity, sickle cell anemia, sarcoid, Stargardt disease, toxoplasmosis, diseases associated with rubeosis, and diseases caused by abnormal proliferation of fibrovascular or fibrous tissue, including all forms of proliferative vitreoretinopathy, whether or not associated with diabetes.

The following examples are intended to further illustrate the present invention, but do not limit it.

### EXAMPLES

### Materials and Methods

### Cell Cultures

Human umbilical vein endothelial cells (HUVECs) were isolated and characterized as described in Caruso A. et al., “HHV-6 infects human aortic and heart microvascular endothelial cells, increasing their ability to secrete proinflammatory chemokines,” J Med Virol. 2002; 67:528-533.

Cells were cultured in endothelial cell growth medium (EGM MV; Promo cell, Heidelberg, Germany) supplemented with 10% (vol/vol) fetal bovine serum (FBS) at 37° C. in a humidified atmosphere including 5% CO₂.

Human lung microvascular endothelial cells (HL-mECs) were purchased from Lonza Clonetics (Walkersville, MD, USA) and cultured in EGM-2 MV growth medium (Lonza, Basel, Switzerland) containing 10% FBS. Adherent cells were grown until 80-90% confluence.

All experiments were performed with cells at passage 2-6.

### Cloning, Production and Nucleofection of Plasmids Expressing the U94 Gene

U94-derived genes were amplified using plasmid U94 pSR2PH as template. PCR products were inserted into the pVAX1 expression vector. Nucleoporation of cells was performed using Amaxa Nucleofector technology (Lonza) following the manufacturer's protocol. Endotoxin-free plasmid expressing U94 or U94-derived genes was added to cells (1×10⁶) resuspended in 100 μl of nucleofection buffer. Experiments were performed at 24 h post nucleofection.

### Tube Formation Assay

Tube formation assays were performed as described in Caccuri F. et al., “Evolution toward beta common chain receptor usage links the matrix proteins of HIV-1 and its ancestors to human erythropoietin,” Proc Natl Acad Sci USA. 2021; 11810.

Briefly, 150 μl of Cultrex Basement Membrane Extract (Matrigel; 10 mg/ml) (Trevigen Inc., Gaithersburg, MD, USA) or reduced growth factor Matrigel (Trevigen Inc.) were transferred to prechilled 48-well culture plates. The plates were then incubated for 1 h at 37° C.

Cells were resuspended in EGM growth medium containing 10% FBS and seeded (5×10⁴ per well). Vessel formation was observed at different times after cells were seeded. Capillary structures were photographed with a Hitachi KP-D50 camera and then quantified as the number of tubes/well.

In some experiments, HUVECs were stimulated with optimal concentrations of human pro-angiogenic molecules such as vascular endothelial growth factor-A (VEGF-A), fibroblast growth factor-2 (FGF-2), or interleukin-8 (IL-8). Experiments were conducted using nucleofected cells or cells stimulated with U94-derived peptides.

### Spheroids Assay

The assay was performed using nucleofected cells or cells stimulated with U94-derived peptides. Spheroids were generated by mixing HUVECs (1.5×10⁵ cells/ml) with 5 mg/ml methylcellulose (Sigma-Aldrich) in EGM growth medium containing 10% FBS, bringing the final volume to 10 ml.

The cells (100 μl/well) were then added to 96-well plates (Greiner Bio-one, Kremsmünster, Austria) and incubated at 37° C., in a 5% CO₂ atmosphere for 24 hours.

Separately, the collagen I gel solution (Rat Tail, Corning) was maintained on ice and neutralized by adding NaOH 0.1 M and PBS 10× to a final pH of 7.4.

Then, 24-well plates were coated with neutralized collagen (200 μl/well) and incubated in a humidified 5% CO₂ incubator for 1 hour at 37° C.

The spheroids from the 96-well plates were collected in Eppendorf tubes and centrifuged at 4000×rpm for 5 to 10 seconds. When a clear pellet was distinguished, the supernatant was removed and the pellet was kept in a volume of about 100 μl of collagen I-neutralized solution.

Each collagen-spheroid mixture was rapidly added to pre-coated 24-well plates at 100 μl/well and incubated for 1 h. After 1 h, 500 μl of different stimuli were added or not to the wells to cover the surface completely and plates were further incubated for 24 h.

Sprouting occurred from the spheroid core, which was photographed with a Hitachi KP-D50 camera, and the sprout number was counted with the spheroids similar sizes from three different wells of the plate.

### SARS-CoV-2 Infection of HL-mECs

Infection experiments were performed using the clinical SARS-CoV-2 isolate AP66 as previously described in Caccuri F. et al., “A persistently replicating SARS-CoV-2 variant derived from an asymptomatic individual,” J Transl Med. 2020; 18:362.

All experiments were performed with a single viral inoculum in a biosafety level 3 (BLS-3) laboratory at an MOI (multiplicity of infection) of 1.

### Statistical Analysis

Data obtained from multiple independent experiments are expressed as mean ± standard deviation (SD). Data were analyzed for statistical significance using the one-way ANOVA, and data were compared using Bonferroni post-test. Differences were considered significant for P <0.05. Statistical tests were performed using GraphPad Prism 8 software.

### Example 1

To evaluate the ability of the peptide of sequence SEQ ID No. 2 to influence the ability of HUVECs to respond to stimulation exerted by different angiogenic mediators, HUVECs were seeded on growth-reduced Matrigel in the absence (NT) or presence of optimal concentrations of different pro-angiogenic stimuli (VEGF-A, FGF-2, or IL-8) alone or in the presence of a control peptide (CTRL) or of the peptide of sequence SEQ ID No. 2 (KDKY).

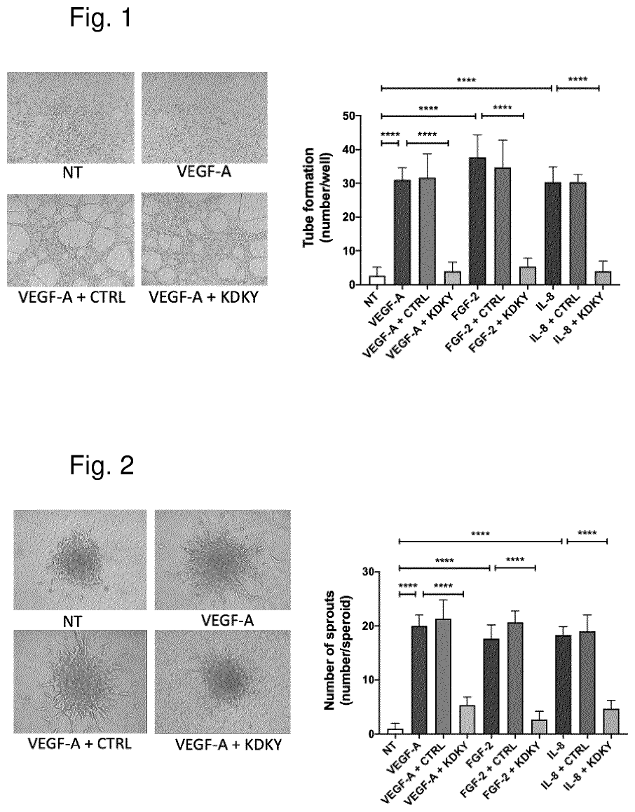

As shown in FIG. 1, untreated HUVECs (NTs) formed a monolayer at 8 hours after seeding on Matrigel. At the same time, HUVECs treated with each of the pro-angiogenic molecules (VEGF-A, FGF-2, or IL-8) migrated and aligned to form tubes organized in a capillary network. This angiogenic activity was not altered by the control peptide (CTRL) and was significantly impaired in cells treated with the peptide of sequence SEQ ID No. 2.

The graph in FIG. 1 illustrates the number of tubes formed in each well. The values represent the mean of one representative experiment out of three with similar results, performed in triplicate. Statistical analysis was performed using the one-way ANOVA test, and the data were compared using the Bonferroni post-test (**** p <0.0001).

### Example 2

HUVEC spheroids embedded in biopolymer gels can be induced to form endothelial sprouts after stimulation with angiogenic factors, thus representing a 3D cell model that mimics in vivo angiogenesis as described in Laib AM, Bartol A, Alajati A, Korff T, Weber H, Augustin HG. Spheroid-based human endothelial cell microvessel formation in vivo. Nat Protoc. 2009; 4: 1202-1215.

On this basis, HUVEC-derived spheroids were incorporated into a type I collagen gel in the absence (NT) or presence of different pro-angiogenic stimuli (VEGF-A, FGF-2, or IL-8) alone or in combination with a control peptide (CTRL) or with the peptide of sequence SEQ ID No. 2 (KDKY).

As shown in FIG. 2, any pro-angiogenic stimulus (VEGF-A, FGF-2, or IL-8) strongly promoted microvessel outgrowth, while the peptide of sequence SEQ ID No. 2 induced a drastic reduction in the sprouting response.

The graph in FIG. 2 illustrates the number of sprouts formed for each spheroid. The values represent the mean of one representative experiment out of three with similar results performed in triplicate. Statistical analysis was performed using the one-way ANOVA test, and the data were compared using the Bonferroni post-test (**** p <0.0001).

These data strongly suggest that the peptide of sequence SEQ ID No. 2 may act as an angiosuppressor by interfering with the mechanisms underlying spontaneous angiogenesis and hampering endothelial cell responses to the stimulation of several potent pro-angiogenic molecules.

### Example 3

SARS-CoV-2-infected HL-mECs secreted a plethora of pro-angiogenic molecules that sustain the capacity of HL-mECs to promote angiogenesis in a in a growth factor-reduced Matrigel.

Indeed, secretoma of SARS-CoV-2-infected HL-mECs is capable of expressing not only VEGF-A and FGF-2, but also several inducers of angiogenesis such as metalloproteinases (MMPs), insulin-like growth factor binding protein-1 (IGFBP-1 insulin growth factor binding protein-1), EGF-like growth factor bound to heparin (HB-EGF, heparin binding--epidermal growth factor), granulocyte-macrophage colony-stimulating factor (GM-CSF), endoglin, angiogenin, and artemin.

In order to understand if the peptide of sequence SEQ ID No. 2 could counteract the angiogenesis induced by the plethora of pro-angiogenic molecules secreted by SARS-CoV-2-infected HL-mECs, experiments were performed by seeding the infected cells on growth factor-reduced Matrigel in the absence or presence of the control peptide (CTRL) or of peptide of sequence SEQ ID No. 2 (KDKY).

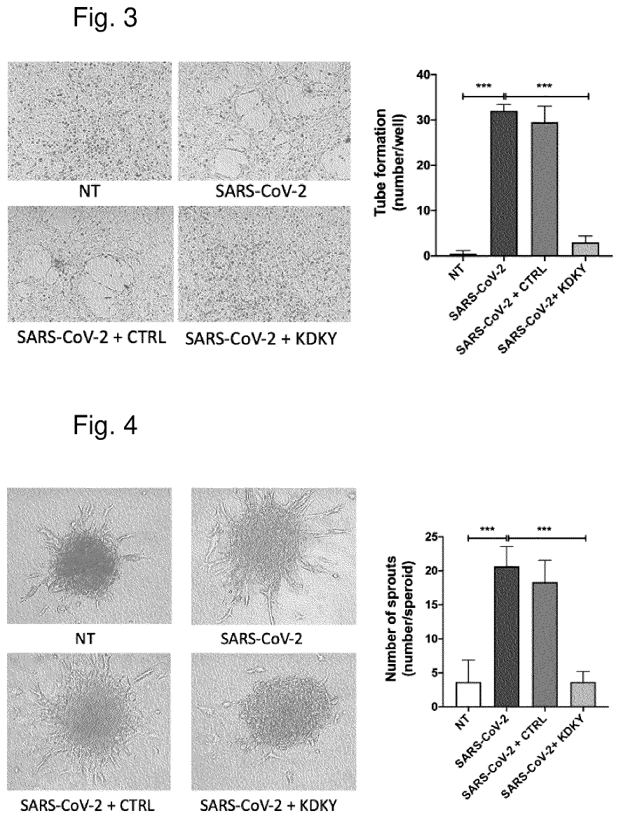

As shown in FIG. 3, in the presence of the peptide of sequence SEQ ID No. 2, SARS-CoV-2-infected HL-mECs did not perform any angiogenic activity. On the other hand, the control peptide CTRL did not interfere with the pro-angiogenic activity induced by SARS-CoV-2 infection.

The graph in FIG. 3 illustrates the number of tubes formed in each well. The values represent the mean of one representative experiment out of three with similar results, performed in triplicate. Statistical analysis was performed using one-way ANOVA test, and data were compared using Bonferroni post-test (**** p <0.0001). NT indicates uninfected HL-mECs.

### Example 4

The anti-angiogenic effect of peptide of sequence SEQ ID No. 2 was also observed in the spheroid assay.

Indeed, as shown in FIG. 4, a dramatic outgrowth of sprouts was observed in SARS-CoV-2-infected spheroids treated or not treated with the control peptide CTRL, whereas the peptide of sequence SEQ ID No. 2 (KDKY) potently inhibited SARS-CoV-2-induced angiogenesis.

The graph in FIG. 4 illustrates the number of sprouts formed for each spheroid. The values represent the mean of one representative experiment out of three with similar results, performed in triplicate. Statistical analysis was performed using one-way ANOVA test, and data were compared using Bonferroni post-test (**** p <0.0001). NT indicates uninfected HL-mECs.

These data confirm the strong and broad anti-angiogenic activity of peptide of sequence SEQ ID No. 2.

### Example 5

In order to understand exactly which amino acids were required to achieve anti-angiogenic activity, four tetra-peptides were synthesized in D configuration, in which each single amino acid of the original peptide KDKY (SEQ ID No. 2) was replaced by an alanine (A), namely ADKY (SEQ ID No. 3), KAKY (SEQ ID No. 4), KDAY (SEQ ID No. 5) and KDKA (SEQ ID No. 6).

HUVECs were seeded on reduced growth factor Matrigel-coated wells wells in complete medium containing 50 ng/ml of VEGF-A or FGF-2 alone or in combination with 10 ng/ml of the control peptide (CTRL), or KDKY, AKDY, KAKY, KDAY or KDKA.

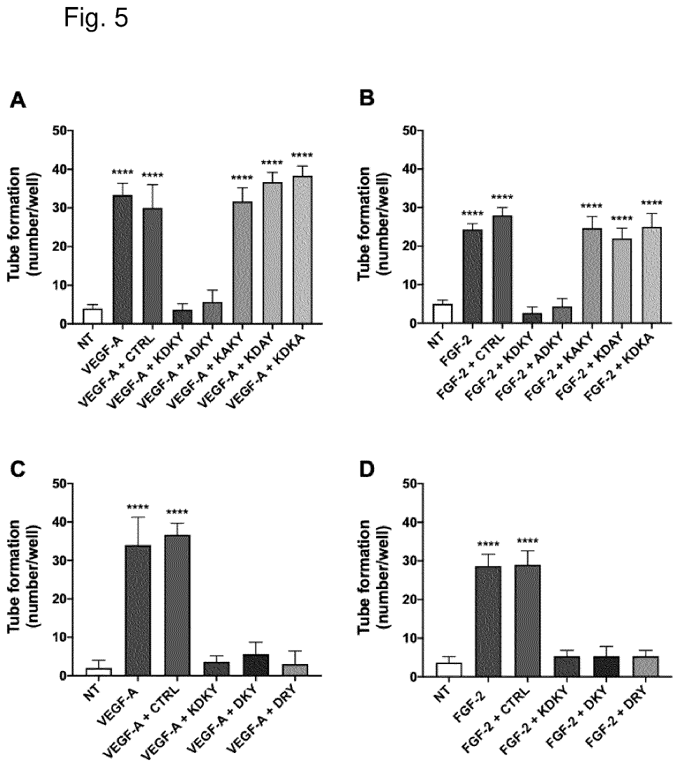

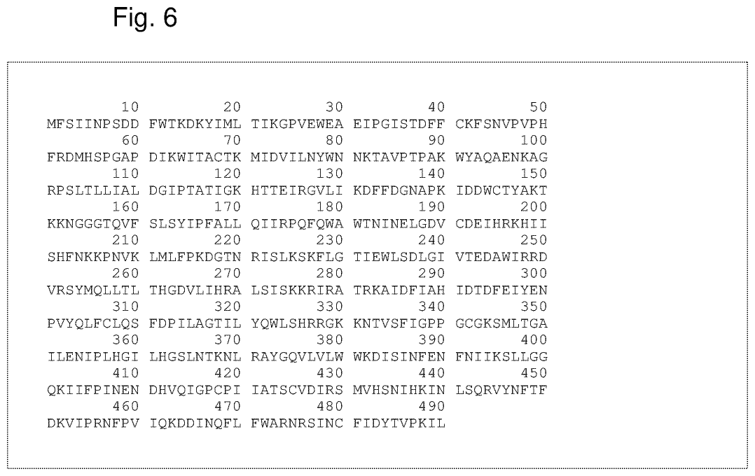

As shown in FIG. 5A-B, only the peptide of sequence ADKY (SEQ ID No. 3) maintained potent anti-angiogenic activity on HUVECs treated with VEGF-A or FGF-2.

Then, DKY sequence peptide and DRY sequence peptide were synthesized and their anti-angiogenic activity was verified as described above.

HUVECs were seeded on reduced growth factor Matrigel-coated wells in complete medium containing 50 ng/ml of VEGF-A or FGF-2 alone or in combination with 10 ng/ml of CTRL, KDKY, DKY or DRY.

As shown in FIG. 5 C-D, both DKY sequence peptide and DRY sequence peptide were able to block angiogenic activity promoted by VEGF-A or FGF-2, as the peptide of sequence KDKY (SEQ ID No. 2).

This result confirmed the importance of the DKY sequence for anti-angiogenic activity and confirmed the usual tolerance of Lys (K) substitution for Arg (R) in biologically active epitopes.

The graphs in FIG. 5 illustrate the number of tubes formed in each well. The values represent the mean of one representative experiment out of three with similar results, performed in triplicate. Statistical analysis was performed using one-way ANOVA test, and data were compared using Bonferroni post-test (**** p <0.0001). NT indicates untreated cells.

LIST OF SEQUENCES
SEQ ID No. 1
MFSIINPSDDFWTKDKYIMLTIKGPVEWEAEIPGISTDFFCKFSNVPVPH
FRDMHSPGAPDIKWITACTKMIDVILNYWNNKTAVPTPAKWYAQAENKAG
RPSLTLLIALDGIPTATIGKHTTEIRGVLIKDFFDGNAPKIDDWCTYAKT
KKNGGGTQVFSLSYIPFALLQIIRPQFQWAWTNINELGDVCDEIHRKHII
SHFNKKPNVKLMLFPKDGTNRISLKSKFLGTIEWLSDLGIVTEDAWIRRD
VRSYMQLLTLTHGDVLIHRALSISKKRIRATRKAIDFIAHIDTDFEIYEN
PVYQLFCLQSFDPILAGTILYQWLSHRRGKKNTVSFIGPPGCGKSMLTGA
ILENIPLHGILHGSLNTKNLRAYGQVLVLWWKDISINFENFNIIKSLLGG
QKIIFPINENDHVQIGPCPIIATSCVDIRSMVHSNIHKINLSQRVYNFTF
DKVIPRNFPVIQKDDINQFLFWARNRSINCFIDYTVPKIL
SEQ ID No. 2
KDKY
SEQ ID No. 3
ADKY
SEQ ID No. 4
KAKY
SEQ ID No. 5
KDAY
SEQ ID No. 6
KDKA
SEQ ID No. 7
XDKY
SEQ ID No. 8
DKYX
SEQ ID No. 9
XDRY
SEQ ID No. 10
DRYX

1. A method for treating a disorder resulting from pathological angiogenesis, comprising administering to a subject in need thereof an effective amount of a peptide of length equal to or lower than 5 amino acids, or a derivative thereof, comprising the sequence DKY, or the sequence DRY.

2. (canceled)

3. The method according to claim 1, wherein said disorder resulting from pathological angiogenesis is selected from the group consisting of a tumor, chronic inflammation, and a neo-vascularisation disorder.

4. The method according to claim 1, wherein said peptide has a length equal to 3 or 4 amino acids.

5. The method according to claim 1, wherein said derivative is a peptide wherein the N-and/or C-terminal is chemically modified or protected with an organic compound selected from the group consisting of phosphoryl (PO₃²⁻), glycosyl, acyl, alkyl, carboxyl, amine, biotin, and ubiquitin.

6. A pharmaceutical composition comprising (a) a peptide of length equal to or lower than 5 amino acids, or a derivative thereof, comprising the sequence DKY, or the sequence DRY, and (b) at least one pharmaceutically acceptable excipient.

7. The pharmaceutical composition according to claim 6, wherein said pharmaceutical composition comprises an amount of said peptide, or a derivative thereof, from 0.0001% to 20% by weight, preferably from 0.0001% to 15% by weight.

8. The pharmaceutical composition according to claim 6, wherein said composition is formulated for oral, parenteral, inhalation (spray, powder or aerosol), topical, rectal, nasal, buccal, vaginal administration or through an implanted device.

9. The pharmaceutical composition according to claim 6, wherein said composition further comprises an antineoplastic agent, preferably chosen from the group consisting of (i) an alkaloid, (ii) an alkylating agent, (iii) an antibiotic or analog, (iv) an antimetabolite, (v) an immunomodulator, (vi) a platinum complex, and (vii) an antineoplastic hormone or analog.

10. A peptide of length equal to or lower than 5 amino acids, or a derivative thereof, comprising the sequence DKY or the sequence DRY.

11. The method according to claim 1, wherein said peptide comprises the sequence XDKY (SEQ ID No. 7) or DKYX (SEQ ID No. 8), or the sequence XDRY (SEQ ID No. 9) or DRYX (SEQ ID No. 10), wherein X is any amino acid.

12. The pharmaceutical composition according to claim 6, wherein said peptide comprises the sequence XDKY (SEQ ID No. 7) or DKYX (SEQ ID No. 8), or the sequence XDRY (SEQ ID No. 9) or DRYX (SEQ ID No. 10), wherein X is any amino acid.

13. The peptide according to claim 10, wherein said peptide comprises the sequence XDKY (SEQ ID No. 7) or DKYX (SEQ ID No. 8), or the sequence XDRY (SEQ ID No. 9) or DRYX (SEQ ID No. 10), wherein X is any amino acid.


SEQUENCE LISTING (6 sequences)

SEQ ID NO: 1 - protein, 490 aa, Human herpesvirus 6
MFSIINPSDDFWTKDKYIMLTIKGPVEWEAEIPGISTDFFCKFSNVPVPHFRDMHSPGAPDIKWITACTKMIDVILNYWNNKTAVPTPAKWYAQAENKAGRPSLTLLIALDGIPTATIGKHTTEIRGVLIKDFFDGNAPKIDDWCTYAKTKKNGGGTQVFSLSYIPFALLQIIRPQFQWAWTNINELGDVCDEIHRKHIISHFNKKPNVKLMLFPKDGTNRISLKSKFLGTIEWLSDLGIVTEDAWIRRDVRSYMQLLTLTHGDVLIHRALSISKKRIRATRKAIDFIAHIDTDFEIYENPVYQLFCLQSFDPILAGTILYQWLSHRRGKKNTVSFIGPPGCGKSMLTGAILENIPLHGILHGSLNTKNLRAYGQVLVLWWKDISINFENFNIIKSLLGGQKIIFPINENDHVQIGPCPIIATSCVDIRSMVHSNIHKINLSQRVYNFTFDKVIPRNFPVIQKDDINQFLFWARNRSINCFIDYTVPKIL

SEQ ID NO: 2 - protein, 4 aa, synthetic construct
KDKY

SEQ ID NO: 3 - protein, 4 aa, synthetic construct
ADKY

SEQ ID NO: 4 - protein, 4 aa, synthetic construct
KAKY

SEQ ID NO: 5 - protein, 4 aa, synthetic construct
KDAY

SEQ ID NO: 6 - protein, 4 aa, synthetic construct
KDKA